(19) United States
(12) Patent Application Publication
(10) Pub. No.: US 2026/0325435 A1
(43) Pub. Date: Oct. 22, 2026

(54) OAK BARK EXTRACT AND COSMETIC USES THEREOF

(71) Applicant: LUCAS MEYER COSMETICS, MASSY (FR)

(72) Inventors: JOAN ATTIA, LAVERNOSE LACASSE (FR); ANNE MANDEAU, TOULOUSE (FR)

(21) Appl. No.: 19/491,076

(22) PCT Filed: Jun. 7, 2024
(86) PCT No.: PCT/EP2024/065703

§ 371 Date: Dec. 8, 2025

(30) Foreign Application Priority Data
Jun. 8, 2023 (EP) 23305911.2

(51) Int. Cl. A61K 8/9783; A61Q 5/00; A61Q 5/02; A61Q 5/04; A61Q 5/06; A61Q 5/10; A61Q 5/12; A61Q 7/00

(52) U.S. Cl. CPC A61K 8/9783; A61Q 5/002; A61Q 5/02; A61Q 5/04; A61Q 5/06; A61Q 5/10; A61Q 5/12; A61Q 7/00

(57) ABSTRACT

The invention relates to the cosmetic use of an oak bark extract as a repairing or regenerating agent of skin appendages or scalp, or as an agent for improving the mechanical or visual properties of skin appendages. The invention further relates to cosmetic compositions comprising an oak bark extract, in particular for cosmetic treatment of skin appendages such as hair and scalp.

### FIELD OF THE INVENTION

The Invention relates to cosmetic active agents useful to treat or improve skin appendages. The Invention also relates to cosmetic methods and cosmetic compositions for skin appendages, in particular for the care of hair and scalp.

### BACKGROUND OF THE INVENTION

The hair is composed of a hair shaft and a root. The hair root is located at the base of the hair follicle in the dermis. The hair follicle ensures the nutrition and protection of the hair shaft and is associated with the sebaceous gland responsible for the production of sebum.

The hair shaft corresponds to the visible part of the hair and has a concentric structure comprising three layers:
The medulla which constitutes the central part of the shaft and which is composed of an amorphous, soft and fatty substance;
The cortex which is the middle layer and the main constituent of the hair. The cortex is responsible for the mechanical properties of the hair. The cortex is made up of keratin and its cells are bound together by an intercellular cement rich in proteins and lipids. It is composed of macrofibrils, which are themselves made of microfibrils containing the helical chains of keratin,
The cuticle which is a thin protective outer layer which is highly keratinized and composed of scale-like cells which overlap one another.

The hair plays a huge role in overall appearance and self-perception.

The hair, and more generally the appendages, are regularly subjected to environmental stress such as wind, pollution, smoke or sun exposure, and to mechanical or chemical treatments. These repeated stress can weaken the skin appendages and even alter their structure. In the case of hair, these repeated stress can alter the cuticle, leading to the lifting and even the detachment of the keratin scales. As a consequence, the visual, mechanical and sensory properties of the hair are altered: The hair becomes rougher, less flexible, difficult to comb, more brittle, finer and less shiny and also less resistant to external stress. Such alterations can be also accentuated with age and scalp can secrete less sebum, have an altered barrier function and be less supple.

As a consequence, the hair care industry has a substantive interest in conceiving innovative care products that can promote hair health and appearance.

There is thus a need for new cosmetic active ingredients to be used in hair care products.

### SUMMARY OF THE INVENTION

The present Invention relates to the cosmetic use of an oak bark extract as a repairing or regenerating agent of skin appendages or scalp, or as an agent for improving the mechanical or visual properties of skin appendages.

More particularly, the present Invention relates to the cosmetic use of a Quercus alba bark extract as an agent for promoting or restoring the skin appendage health, for instance for revitalizing the scalp and/or the hair.

In certain embodiments, the oak bark extract is a water extract or a hydro-alcoholic extract of oak bark. Preferably, the oak bark extract is a hydro-alcoholic extract from Quercus alba bark.

In some embodiments, the oak bark extract is used to increase hair density and/or to decrease or prevent grey hair. In particular, the oak bark extract is used to slow-down hair cycle.

In some embodiments the oak bark extract is used for improving the strength, the resistance and the shine of skin appendages.

In other embodiments, the oak bark extract is used for repairing the keratin structure of skin appendages.

In another embodiment, the oak bark extract is used as a sebo-regulator agent of the scalp or as an agent for improving the barrier function and/or the suppleness of scalp. In a particular embodiment, the oak bark extract is used to promote the sebum production, in particular to increase and/or improve the quality of sebum production in dry scalp. The oak bark extract can be also used to sooth and/or normalize dry scalp.

For instance, the oak bark extract is used to prevent, alleviate, reduce or treat one or several non-pathological modifications of the skin appendages selected from a loss of thickness, a loss of softness, an increase in dryness, an increase in roughness, a decrease in shiny or silky appearance, a dull or lifeless appearance, a decrease in strength, a decrease in mechanical resistance, brittle skin appendages, a decrease in hair density, a decrease in hair volume, unruly hair or hair more difficult to comb or to shape and combinations thereof.

In a particular embodiment, the oak bark extract is used in combination with phospholipids such as lecithin. Preferably, the oak bark extract is used in combination with phospholipids for repairing damaged African-type hair.

In some embodiments, the skin appendages are selected from the group consisting of eyelashes, eyebrows, beard, mustache, hair and combinations thereof. Preferably the skin appendages are hair, in particular damaged, weakened or stressed hair such as dyed hair or bleached hair. Typically, the oak bark extract is applied on the skin appendages in the form of a cosmetic product.

In some embodiments, the bark extract, preferably the Quercus alba bark extract, is obtained by extraction with hydroalcoholic extraction, preferably at a temperature from 30 °C to 70 °C. The extraction solvent may be, for instance, H₂O/EtOH in a volume ratio from about 0.20 to about 5.0.

In a further aspect, the Invention relates to an oak bark extract as described herein, in particular to a hydro-alcoholic bark extract from Quercus alba as described herein and a process of manufacturing as described herein.

In an additional aspect, the Invention relates to a cosmetic ingredient to be used in a cosmetic product for skin appendages comprising an oak bark extract as defined herein in a vehicle preferably selected from glycerol, C₂-C₅ alkanediols, ketal esters, water, and mixtures thereof. A preferred vehicle is water/glycerin mixture, e.g a water/glycerin mixture in a weight ratio of about 0.1 to about 0.5, such as about 0.25.

In another aspect, the Invention relates to a cosmetic product for repairing or regenerating skin appendages or scalp, for promoting or restoring the scalp and/or hair health or for revitalizing the scalp and/or hair or for improving the visual or mechanical properties of skin appendages comprising an oak bark extract as defined herein, preferably a Quercus alba bark extract as defined herein, as a cosmetic active agent with one or more cosmetically acceptable ingredients.

The cosmetic product can be a hair care product preferably selected from the group consisting of a shampoo including a dry or solid shampoo, a conditioner, a balm, a mask, an oil, a serum, a lacquer, a hair fixing gel, a hair spray, a perming product, a hair coloring product and an antiparasitic hair product.

In an additional aspect, the Invention relates to a cosmetic method for repairing or regenerating skin appendages or scalp, for promoting or restoring the scalp and/or hair health or for revitalizing the scalp and/or hair or for improving the visual or mechanical properties of skin appendages, said method comprising applying an oak bark extract as defined herein, preferably a Quercus alba bark extract as defined herein, a cosmetic product as defined herein on the skin appendage.

Each of the aspects and embodiments described herein are capable of being used together, unless excluded either explicitly or clearly from the context of the embodiment or aspect.

### FIGURES

FIG. 1 shows the effects of Effects of an White Oak Bark extract of the invention (white oak 0.05%) on Human hair follicular dermal papilla cells (HFDPc) proliferation as compared to untreated cells (“Untreated”-namely cells incubated without the extract in serum-free medium) and positive group (cells incubated with serum-supplemented medium).

FIG. 2 shows the effects of a White Oak Bark extract (0.05%) of the Invention on sebum production by sebocytes versus untreated cells and positive control (oleic acid at 30 μM).

FIG. 3 shows the apoptotic hair bulbs percentage upon time for hair follicles treated with a White Oak Bark extract of the Invention (white oak 0.05%) versus untreated hair follicles.

FIGS. 4A, 4B, 4C and 4D show the results obtained in Example 6 in terms of glow, hair elongation, hair strength and hair friction (hair harmony assay) for hair tresses from different hair types (Caucasian, Asian and African hair types) and artificially damaged by bleaching and treated with a conditioner containing a White Oak Bark extract of the Invention (WO) as compared to placebo (tresses treated with a conditioner devoid of White Oak Bark extract.

FIGS. 5A and 5B show the results obtained in Example 7, in terms of hair force and hair friction (hair harmony assay), of the combination of white oak bark extract and sunflower lecithin (WO+SL) versus white oak bark extract alone or sunflower lecithin (SL) alone (as compared to placebo) on artificially damaged African-type hair.

### GENERAL DEFINITIONS

As used herein, the singular forms “a”, “an”, and “the” include plural referents unless the context clearly dictates otherwise. For example, “a” or “an” include “at least one” and “one or more.”

The terms “comprising”, “comprises” and “comprised of” as used herein are synonymous with “including”, “includes”, “containing”, “contains”, and grammatical variants thereof, are inclusive or open-ended and do not exclude additional, non-recited members, elements, or method steps.

The term “about” refers to a range of −10% to +10% of the numerical value, unless the term is otherwise specifically defined in context.

### DETAILED DESCRIPTION OF THE INVENTION

In traditional medicine, bark from Quercus species has been used topically for the treatment of burns and wounds, and orally for the treatment of gastrointestinal diseases. Barks from Quercus species are known to contain large amounts of polyphenolic compounds, which may exhibit biological activities such as anti-inflammatory and anti-bacterial activities. Cosmetic uses of certain oak bark extracts such as Quercus robur extract have been suggested in the prior art.

The Applicant conceived a particular extract from a Quercus species, namely Quercus alba (also called herein white oak). Surprisingly, the Applicant showed that this oak bark extract displayed pleiotropic actions on both the hair and the scalp, which go beyond the mere possible anti-inflammatory and/or antioxidant activities suggested in the prior art. Surprisingly, the white oak bark extract was shown to be effective on every hair type.

The Applicant showed that the oak bark extract can exert multiple actions resulting in an improvement of the overall health and properties of both the scalp and the hair.

Indeed, the oak bark extract of the invention was shown to exert seboregulator activity on sebocytes in vitro (Example 2). More precisely, the oak bark extract was able to significantly improve the production of lipid droplets in sebocytes in the absence of oleic acid induction in vitro. The oak bark extract was also shown to improve the quality of sebum production by exerting a protective action against lipoperoxidation.

The Applicant further showed that the oak bark extract slowed down the entry of hair bulbs into apoptosis in an ex vivo model of human hair follicle culture, and thus can increase hair longevity and anchorage (Example 3). In vitro, this effect was confirmed on the apoptotic markers caspases 3 and 7, which labelling was reduced in presence of the oak bark extract (Example 4).

At last, the Applicant further showed that the oak bark extract of the invention can act as a hair repairing agent and can improve the mechanical and visual properties of hair.

As illustrated in Example 6 and Example 7, which relates to an ex vivo assay on artificially damaged hair strands, the oak bark extract of the Invention is able to increase the hair shine, resistance and strength and to repair hair structure, regardless hair ethnic origin and more generally regardless hair type, e.g. as defined by the hair typing system developed by Andre Walker. A synergic effect of the oak bark extract when used in combination with lecithin was further evidenced in terms of strength and friction on artificially damaged African-type hair. At last, as evidenced in Example 8, the Applicant performed a clinical study which showed that the white oak extract of the invention improves the health of the hair and scalp in vivo. When applied chronically, the white oak bark extract of the invention increased the hair density and decrease the proportion of grey hair as compared to the placebo group. The hair brightness was also increased with the group treated with the extract of the invention as compared to the placebo group. The extract of the invention also improved scalp health, in particular by soothing dry scalp. Thus, the oak bark made the hair look stronger, younger and healthier. Thus, the oak bark extract of the invention exerts pleiotropic actions resulting in an overall improvement of the health and the aspect/appearance of the hair and the scalp. Thus, the oak bark extract of the invention finds applications as a cosmetic active ingredient in skin appendage care products, e.g. as an agent to promote skin appendage health, as an agent to revitalize skin appendages, as a repairing or regenerating agent or as an agent to improve the mechanical and visual properties of skin appendages, in particular hair.

Accordingly, in certain aspects, the Invention relates to an oak bark extract, preferably obtained from Quercus alba.

The Invention further relates to a cosmetic ingredient or a cosmetic product comprising said extract as a cosmetic active agent.

The Invention further relates to the use of said extract as a cosmetic active agent in cosmetic products for skin appendages, in particular in hair care products.

In a particular aspect, the Invention relates to the use of an oak bark extract as a cosmetic agent to improve the health, the quality and/or the longevity of skin appendages, in particular hair. In another particular aspect, the Invention relates to the use of an oak bark extract as a repairing or regenerating agent of skin appendages or scalp, and/or as a sebo-regulator agent of the scalp and/or as an agent for improving the physical or visual properties of skin appendages such as shine, strength and resistance.

In some aspects, the bark oak extract of the invention is used to promote the well aging of the hair and the scalp.

The Invention further relates to a cosmetic treatment of skin appendages and/or scalp comprising topically applying an oak bark extract, preferably from Quercus alba, as such or in the form of a cosmetic product, on skin appendages and/or scalp.

The uses and methods described herein are preferably non-therapeutic and cosmetic. The uses and methods described herein are preferably performed on healthy skin appendages and/or healthy scalp.

### The Oak Bark of the Invention

As used herein, an “oak bark extract” refers to an extract obtained from the bark (also called the cortex) of Quercus species.

The Quercus genus belongs to Fagaceae family and encompasses various species such as Quercus robur, Quercus petraea, Quercus rubra, Quercus pubescens, Quercus alba, Quercus x rosacea and Quercus pyrenaica, and Quercus peduncularis.

In certain embodiments, the bark extract of the Invention is selected from the group consisting of Quercus robur, Quercus rubra, Quercus alba and combinations thereof. In preferred embodiments, the extract of the invention is from bark of white oak, also called Quercus alba.

The bark is typically provided as a byproduct or coproduct of wood industry.

The oak bark extract of the invention can be prepared by any methods known by the skilled artisan. Such methods include distillation, extraction with sub-critical or supercritical fluid such as supercritical CO₂ or subcritical water, solid-liquid extraction, maceration, decoction and the like.

In some embodiments, the bark extract is obtained by solid-liquid extraction using a solvent selected from water, organic solvents and mixtures thereof. Appropriate organic solvents encompass, without being limited to, polyols such as glycerin and alkanediols, e.g. C₂-C₅ alkanediols such as ethylene glycol and 1,3-propanediol, lower alcohols and combinations thereof.

In a particular embodiment, the oak bark extract is obtained by solid-liquid extraction using a H₂O/solvent mixture wherein the solvent is selected from C₂-C₅ alcohol, C₂-C₅ alkanediols, glycerin and mixtures thereof. The weight ratio of the water to the solvent is typically from 0.1 to 10 such as 0.25 to 4.0.

In a more particular embodiment, the oak bark extract is obtained by hydro-alcoholic extraction, namely by using a mixture of water and lower alcohol (typically C₂-C₅ alcohol) as extraction solvent. C₂-C₅ alcohols of interest encompass, without being limited to, ethanol (EtOH), propanol, butanol, pentanol, isomers thereof such as isopropanol, isobutanol or tert-butanol, and combinations thereof.

A solvent of interest is for instance H₂O/EtOH mixture in volume ratio from about 0.10 to about10.0 such as from about 0.20 to about 5.0, from about 0.30 to about 3.0 or from about 0.30 to about 1.0.

In some embodiments, the oak bark extract is obtained by extraction of the bark with H₂O/EtOH in a volume ratio from 0.25 to 0.75, for instance from 0.40 to 0.60 such as about 0.50.

For illustration only, the oak bark extract, preferably from Quercus alba, can be obtained as illustrated in Example 1.

In some embodiments, the oak bark extract of the invention, preferably from Quercus alba, is characterized in that it comprises:
from 1.0% to 7.0%, preferably from 2.0% to 6.0% of flavonoids expressed in rutin (AlCl3 method e.g. as described in Shraim et al., Food Science and Technology 2021, 150, 111932), and
from 20% to 40%, preferably from 25% to 35% of total carbohydrates expressed in glucose (Phenol method e.g. as described in Dubois et al., Analytical Chemistry 1956, 28, 350-356.).

In some further embodiments, the oak bark extract may further comprise:
from 5% to 25%, preferably from 10% to 21% or from 15% to 21% of polyphenols expressed in gallic acid (FOLIN-CIOCALTEU reagent method e.g. as described in: Singleton et al., Am. J. Enol. Vitic. 1965, 16, 144-158), and
from 0.5% to 7.5%, preferably from 1.0% to 6.5% or from 2.5% to 6.5% of proanthocyanidins expressed in procyanidin B2 (Porter's method as described in Porter et al., Phytochemistry 1986, 25, 223-230).

It goes without saying that the oak bark of the Invention is adapted for a cosmetic use when used in appropriate amounts, namely exerts cosmetic effects without any significant toxicity or side-effects.

In a further aspect, the Invention relates to a method for preparing the oak bark extract comprising the steps of:
a) Extracting an oak bark with a solvent preferably as described herein, in particular with water, organic solvents and mixtures thereof as described herein, and
b) Recovering the liquid phase from step b) and at least partially removing the solvent so as to obtain an oak bark extract.

Before step a), the oak bark, e.g. collected as a coproduct or byproduct of wood industry, preferably Quercus alba bark, can be subjected to one or several treatments so as to be in a state suitable for performing the step a) of extraction. For instance, the oak bark can be subjected to one or several treatments such as washing, drying, grinding, milling, freeze-drying, freeze-grinding, freezing/thawing and combinations thereof.

In some preferred embodiments, the bark is dried and milled before being extracted in step a).

In a particular embodiment, the oak bark is provided in the form of a dried and milled bark preferably from Quercus alba for performing step a).

Step a) is preferably performed by using a solvent selected from water, C₂-C₅ alcohol, C₂-C₅ alkanediols, glycerin and mixture thereof. In a particular embodiment, step b) is a hydro-alcoholic extraction e.g, namely a liquid-solid extraction using a H₂O/EtOH mixture, such as a H₂O/EtOH with a volume ratio from 0.25 to 0.75 or from 0.40 to 0.60, e.g. about 0.50, as extraction solvent.

In some embodiments, the ethanol used in the process according to the invention is preferably a bioethanol, i.e. a biobased ethanol. Bioethanol may be obtained by enzymatic hydrolysis of starch originating from cereals, in particular from maize, or by fermentation starting with plants containing sucrose. In step b), the oak bark is typically submerged in the solvent. The oak bark is macerated in the solvent at room temperature or under heating, e.g. at a temperature from 40 °C to 70 °C, e.g. at about 50 °C.

The duration of the extraction step may vary according to the plant material (herein the oak bark), the amount of the plant material, and the extraction solvent.

Typically, the extraction step can last from few minutes to several hours, e.g. from 5 min to 4 hours or from 30 min to 2 hours such as 45 min, 60 min or 75 min.

In some embodiments, the extraction can be performed with stirring or under sonication or microwaves, to facilitate the release of the molecules of interest from the bark.

In some other embodiments, the extraction is performed without stirring and/or ultrasonication. Step b) can be performed for instance in a reactor fitted with a condenser and with a heating means. Alternatively, the extraction step can be performed by percolation, in particular under high pressure.

At the end of the extraction step, the plant material (herein the oak bark, preferably the Quercus alba bark) and the liquid phase (herein the solvent charged with the molecules of interest from the bark) are separated. This separation may be performed by any method known to the skilled in the art, such as pressing, decantation, filtration or centrifugation. Preferably, it is performed by filtration.

Step b) can be repeated at least one time (e.g. 1, 2 or 3 times) on the recovered bark to optimize the extraction yield.

The recovered liquid phase is then concentrated e.g. under reduced pressure or under vacuum so as to eliminate the organic solvent. If needed, the concentrated phase is subjected to a drying step, such as spray-drying so as to eliminate residual water. The oak bark extract is obtained typically in the form of a powder, which can be directly used.

For sake of clarity, it should be noted that the term “oak bark extract” refers to the product, namely the mixtures of molecules, obtained by extraction of oak bark after the removal of the possible solvent and water used in the extraction process.

In some embodiments, the product obtained in step c) corresponds to the final oak bark extract, which can be directly used according to the invention.

In some other embodiments, the product obtained in step c) is an extract precursor which is subjected to one or several additional steps before being used as an oak bark extract. Such additional step(s) may be a drying step e.g. on an appropriate support such as maltodextrin, and/or an additional purification step such as a distillation step, for example molecular distillation or fractional distillation, a precipitation step, a filtration step such as ultra-filtration, an extraction step, notably liquid-liquid extraction and the like. The additional purification step(s) may be performed e.g. to remove coloring.

Enzymatic post-treatment e.g. by means of tannase or cellulase may also be performed in order to facilitate further filtration steps, ameliorate solubility and stability of the final oak bark extract.

In some embodiments, the extract obtained in step c) is subjected to a formulation step. For example, this step of formulation may comprise adding one or more cosmetically acceptable excipients, in particular a cosmetically acceptable vehicle or support, e.g. so as to give a cosmetic ingredient e.g. to be used in a more complex cosmetic product.

For example, the oak bark extract according to the invention may be diluted in a suitable organic or plant solvent, for example glycerol (also called glycerin), alkanediols such as propylene glycol, butylene glycol, pentylene glycol, ketal esters e.g. ketal levulinates such as methyl or ethyl levulinate or glycerol ketal levulinate, water, and mixtures thereof, encapsulated in a vectorization system, for example liposomes, or alternatively absorbed onto a solid vehicle such as maltodextrin. For instance, the bark oak extract, preferably hydro-alcoholic oak bark extract from Quercus alba, is dissolved in a mixture of glycerol and water. Antioxidant and preservative-like compounds (namely compounds having some antibacterial or bacteriostatic activities) such as caprylyl glycol or glycerol caprylate can be also added.

As a further example, the bark oak extract, preferably a hydro-alcoholic bark oak extract from Quercus alba, can be formulated as an emulsion with an appropriate plant oil and phospholipids, e.g. with sunflower seed oil as oil phase and sunflower lecithin as emulsifier.

Such a step of formulation results in a cosmetic ingredient which is also part of the Invention. In some embodiments, the cosmetic ingredient comprises an oak bark extract in combination with a carrier which is glycerin or a water/glycerin mixture. The amount of the oak bark extract in the cosmetic ingredient is at least or at least about 0.5%, 1%, 5%, 10%, or more by weight. The amount of the oak bark extract in the cosmetic ingredient is typically from 0.5% to 10% by weight, such as 1% to 5.0% by weight, e.g. 2.5% by weight. Such a cosmetic ingredient can be used as such or incorporated in a cosmetic composition, typically in an amount of 0.1% to 5.0% by weight, as described herein.

### Cosmetic Uses and Cosmetic Methods

In a particular aspect, the oak bark extract of the invention, such as Quercus alba bark extract, is used as a cosmetic agent for the care of skin appendages and/or scalp. As used herein, “skin appendages” refer to epidermal and dermal-derived components of the skin that include hair, nails, and sebaceous glands. Skin appendages do not include skin or mucous.

In some embodiments, skin appendages are selected from the group consisting of eyelashes, eyebrows, beard, mustache, hair, nails and combinations thereof. In a particular embodiment, the skin appendages refer to keratin fibers such as eyelashes, eyebrows, beard, mustache, and hair, preferably hair, beard or mustache, and more preferably hair.

In the context of the Invention, the skin appendages or the scalp are preferably healthy, which means that the skin appendages or the scalp are/is not afflicted with any disease, disorder or wound such as infections caused by fungi or bacteria, eczema, dermatitis, insect bites, alopecia areata, and the like. Preferably, the oak bark extract of the Invention does not exert any therapeutic effect when applied on the skin appendages or the scalp when used according to the Invention.

The skin appendages or the scalp may be weakened, damaged, and/or stressed e.g. because of normal biological phenomena related to age or because of treatment or exposure to an external insult which has weakened them, i.e. has made them more susceptible to aggression and/or subsequent treatments, or damaged them e.g. by decreasing the strength or the visual aspect of the skin appendages, without causing any pathology or wound.

By way of example, the Applicant has shown that bleaching alters the shine, the softness and the strength of hair.

As another example, the oak bark extract can be used on age-related weakened or damaged hair or scalp.

As used herein, “a cosmetic agent” or “a cosmetic active agent” refers to an agent exerting a cosmetic effect on the skin appendages or the scalp.

As used herein, “a cosmetic effect” refers to any non-therapeutic effect, aiming at modifying and/or improving the appearance or the physical properties of the scalp or the skin appendages such as eyelashes, eyebrows, beard, mustache, hair and combinations thereof. The cosmetic effect may help to increase the resistance of the scalp or the skin appendages to external insults (such as sun exposure, wind, humidity, dryness, chemicals, smoke, pollution, and/or cosmetic treatments), or prevent, alleviate, reduce, slow-down, delay the onset, and/or correct a non-pathological alteration or modification of the properties of the skin appendages or the scalp, e.g. resulting from exposure to such external aggressions or related to the age.

Non-pathological modifications of skin appendages encompass, without being limited to, a decrease in shine, a skin appendage more brittle, a decrease in mechanical resistance or in strength, a decrease in softness, a decrease in thickness, a decrease in hair density and the like. Non-pathological modifications of scalp encompass, without being limited to, a decrease in suppleness, an increase in dryness, a decrease in sebum production, a loss of sebum production quality, a decrease in scalp barrier function, and the like.

In a particular aspect, the cosmetic effect may refer to a well aging effect, namely an effect aiming to reduce, lighten or improve the modifications related to age without exerting any active anti-aging action.

In another particular aspect, the oak bark extract of the invention may be used on damaged, stressed and/or weakened skin appendages and/or scalp.

For illustration only, the damaged, stressed or weakened skin appendages or scalp may have been subjected to a treatment causing non-pathological modifications of the properties or the appearance of the skin appendage or scalp. Such treatments can be physical or chemical. Such treatments encompass, without being limited to, any cosmetic treatments causing mechanical and/or thermal stress such as drying, brushing, straightening, curling (by use e.g. curling iron, straightener, hair dryer, brushing with a brush or comb) or any cosmetic treatments causing chemical stress, e.g. treatments in which a product, in particular cosmetic product, is applied on the skin appendages. For illustration, one can refer to frequent washing with a shampoo and/or a conditioner, the application of a leave-on product such as a lotion, a lacquer, an oil, a gel fixative, a make-up product or any other cosmetic product, in particular a hair product including those used for making permanent waves, antiparasitic products such as anti-lice products, coloring or bleaching products.

As a further illustration only, the damaged, stressed or weakened skin appendages or scalp may have been subjected to an environmental stress such as sun exposure, pollution, smoke, dust, wind, dryness, seawater, chlorinated water, humidity and combinations thereof.

In some embodiments, the skin appendages are hair which have been subjected to an external stress, in particular to a cosmetic treatment e.g. as described herein.

As illustrated in Example 6, the oak bark extract of the Invention is able to increase the shine and the strength in weakened or damaged hair strands and to repair their keratin structure whereby the hair roughness is decreased, regardless the hair type.

As illustrated in Example 3, the oak bark extract of the Invention further slows down the entry of hair bulbs into apoptosis in an ex vivo model of human hair follicle culture, and thus can increase hair longevity and anchorage.

Accordingly, the oak bark extract of the Invention can be used as a repairing agent or a regenerating agent of skin appendages and scalp, preferably of hair and/or scalp.

In a particular aspect, the oak bark extract of the Invention can be used to restore or improve the visual or mechanical properties of skin appendages, in particular hair.

In the clinical study of Example 8, the oak bark extract of the Invention was shown to increase hair density by slowing-down the hair cycle (and by the way the hair loss), even during the summer when the hair fall is naturally increased. The oak bark extract was also shown to decrease or prevent white/grey hair. Accordingly, the oak bark extract can be used as an agent to increase or replenish hair density and/or prevent or decrease white/grey hair. In a more general aspect, the oak bark extract can be used to make the hair look younger and revitalized.

The oak bark extract can be used on every hair type.

Hair can be defined through ethnic origin. There are three ethnic-type hair: namely Asian type hair, Caucasian type hair and African type hair. Regardless their types, all hair have the same structure as described in the background section.

Hair can be also classified through the hair typing system developed by André Walker in 1997. It was first developed to promote hair care products. This system defined four hair types corresponding to their original appearance: straight as type 1, wavy as type 2, curly as type 3, and coily as type 4. Although each category was very simple at first, over the years, the subcategories a, b, and c were gradually created to classify all hair types further within each group. Thus, each hair type is thus sub-divided into a, b, and c. Asian-type hair generally corresponds to hair type 1 (straight hair) as defined in the hair typing system. Caucasian-type hair generally corresponds to hair type 2 (wavy hair), as well as some subclasses in hair type 1 (straight hair) and hair type 3 (curly hair). African-type hair generally corresponds to hair type 3 (curly hair) and 4 (kinky hair).

As used herein a “regenerating agent” refers to a cosmetic agent able to exert one or several cosmetic effects aiming at restoring or boosting the health of the skin appendages or the scalp. For instance, a “hair regenerating agent” may exert one or several cosmetic effects such as increasing hair thickness, replenishing hair density, increasing hair longevity, preventing or reducing hair loss, increasing hair growth, increasing or improving hair anchorage and combinations thereof. Preferably, the oak bark extract of the invention replenishes hair density by slowing-down hair life cycle.

As used herein a “repairing agent” refers to a cosmetic agent able to exert one or several cosmetic effects aiming at alleviating, reducing, correcting or treating one or several non-pathological modifications of the skin appendages and/or aiming at repairing the appendages' structure. In such use, the appendages can be damaged, weakened and/or stressed. For instance, a “hair repairing agent” can exert one or several cosmetic effects selected from restoring or improving the hair shine, strength, and/or mechanical resistance as well as repairing hair structure, in particular hair keratin structure.

As used herein, the “mechanical properties of skin appendages, in particular those of hair” encompass, without being limited to, their strength, their flexibility, their resistance, their brittle tendency, their softness, their smoothness, their ability to be shaped or combed, their thickness and combinations thereof. The mechanical properties of keratin fibers, in particular the hair, can be measured through elongation test as illustrated in the Example section (e.g. in order to determine the resistance of hair to elongation) or by sensory test (e.g. tactile assay to assess softness).

The cuticle present on the surface of skin appendages such as keratin fibers, in particular the hair, has an impact on their visual and mechanical properties. In fact, if the cuticle is damaged, the scales of the keratin fibers, in particular the hair, rise and may even become detached. As a consequence, the keratin fibers become dull, friable, brittle, and difficult to shape. As used herein, the “visual properties of skin appendages, in particular those of keratin fibers such as hair” encompass, without being limited to, the shine or glossy aspect, the color and combinations thereof.

The visual aspect of skin appendages can be assessed by sensory visual test or by assay such as glow measurement as illustrated in Example 6 or by color measurement.

The structure integrity of keratin fibers can be assessed, for instance, by hair symphony assay as illustrated in Example 6.

In another aspect, the oak bark extract is used to prevent, to delay the onset of, protect against, alleviate, reduce, correct or treat one or several non-pathological modifications of the skin appendages such as a loss of thickness, a loss of softness, an increase in dryness, an increase in roughness, a decrease in shiny or silky appearance, a dull or lifeless appearance, in particular a dull hair color, a decrease in strength, a decrease in mechanical resistance, brittle skin appendages, a decrease in hair density, a decrease in hair volume, unruly hair or hair more difficult to comb or to shape and combinations thereof.

In a particular aspect, the oak bark extract can be used for improving the mechanical and/or visual aspects of skin appendages, in particular the strength, the resistance, the softness and/or the shine of skin appendages. Preferably, the skin appendages are keratin fibers such as hair, in particular damaged, stressed and/or weakened hair.

In a further embodiment, the oak extract of the Invention is used to improve the appearance (e.g. the visual properties) of skin appendages such as keratin fibers.

For instance, the oak bark extract of the Invention is used to improve or restore the shine or the silky appearance of hair, or to provide the hair with a healthy or beauty appearance. As illustration only, the oak bark extract can be used to provide the hair with a healthier, younger and stronger looking appearance.

In an additional aspect, the oak bark extract of the Invention can be used as a protecting agent of skin appendages and/or scalp. For instance, the oak bark extract can be used for protecting skin appendages and/or scalp against an external stress such as environmental stress (e.g. sun exposure, pollution, smoke or dust exposure, chemical exposure, wind, dryness, seawater, chlorinated water, humidity and combinations thereof) or a cosmetic treatment as described herein, such external stress potentially causing one or several non-pathological modifications of the skin appendages or scalp.

In another embodiment, the oak bark extract of the Invention is used to improve the mechanical properties of hair, e.g. to improve or restore the hair softness, to decrease hair roughness, to increase hair strength and resistance e.g. against elongation, to restore hair structure, to increase hair thickness and/or density, to reduce hair breakage and/or hair forks, to make the hair easier to comb and combinations thereof.

As illustrated in Example 2, the oak bark extract of the Invention is able to induce the sebum secretion in sebocytes in the absence of oleic acid induction. Of note, such an increase in sebum secretion is dampened in the presence of oleic acid.

In a particular aspect, the Invention relates to the use of an oak bark extract as a seboregulator agent of the scalp, in particular for improving sebum production, e.g. in quantity and/or quality, in scalp, preferably in dry scalp.

The oak bark extract of the Invention can be used to improve or restore the barrier function of the scalp, and/or to improve the suppleness of scalp, in particular that of dry scalp. In an additional aspect, the oak bark extract of the invention can be used as a moisturizing agent of scalp.

In a further particular aspect, the oak bark extract of the invention can be used to restore or improve sebum production in dry scalp, e.g. in scalp with age-related sebum production deficit.

As illustrated in Example 7, the Applicant surprisingly showed that the cosmetic effects of the oak bark extract can be potentiated when used in combination with phospholipids e.g. such as lecithin, in particular sunflower lecithin, on hair belonging to hair type 3 or 4, such as African type hair.

Thus, in some aspects, the oak bark extract of the Invention is used in combination with phospholipids. Typically, the phospholipids such as sunflower lecithin can be used as an emulsifier agent in combination with oil such as sunflower seed oil (e.g. to solubilize the oak bark extract in the form of an emulsion). The lecithin can also act as an agent to potentiate the action of the oak bark extract on the skin appendages or the scalp, in particular to potentiate the repairing effects of the oak bark extract on damaged type 3 or 4 hair such as African-type hair.

The weight ratio of the oak bark extract to the phospholipids may be at least or at least about 0.01, 0.05, 0.1, 0.5, or 1,0. The weight ratio of the oak bark extract to the phospholipids may be from 0.01 to 1,0, preferentially from 0.02 to 0.2.

In some other embodiments, the oak bark extract is used in combination with lecithin. In another embodiment, the oak bark extract is used in combination with lecithin such as sunflower lecithin and a plant oil preferably selected from olive oil, rape oil, almond oil, jojoba oil, avocado oil, coconut oil, wheat germ oil, sunflower seed oil, soybean oil and combinations thereof. In a particular embodiment, the oak bark extract is used in combination with sunflower lecithin and sunflower seed oil. In the uses and methods described herein, the oak bark extract of the invention is preferably applied topically, generally by means of a cosmetic composition. In other words, the oak bark extract of the invention is generally formulated into a cosmetic composition before being applied on the scalp or on the skin appendages. Said cosmetic composition may further comprise phospholipids such as lecithin e.g. as a potentiator agent and/or an emulsifier agent. In some embodiments, the oak bark extract of the Invention is present in the cosmetic composition in an amount of at least or at least about 0.0001%, 0.001%, 0.01%, 0.1%, 1%, 5%, or 10% in weight relative to the total weight of the cosmetic composition. The oak bark extract of the Invention is generally present in the cosmetic composition in an amount of at least or at least about 0.0001% in weight relative to the total weight of the cosmetic composition. The oak bark extract of the Invention is generally present in the cosmetic composition in an amount of at least or at least about 0.001% in weight relative to the total weight of the cosmetic composition. The oak bark extract of the Invention is generally present in the cosmetic composition in an amount of at least or at least about 0.01% in weight relative to the total weight of the cosmetic composition. The oak bark extract of the Invention is generally present in the cosmetic composition in an amount of at least or at least about 0.1% in weight relative to the total weight of the cosmetic composition. The oak bark extract of the Invention is generally present in the cosmetic composition in an amount of at least or at least about 1% in weight relative to the total weight of the cosmetic composition. The oak bark extract of the Invention is generally present in the cosmetic composition in an amount of at least or at least about 2% in weight relative to the total weight of the cosmetic composition.

The oak bark extract of the Invention is generally present in the cosmetic composition in an amount from about 0.0001% to about 10%, preferably from about 0.001% to about 5.0% such as from about 0.01% to about 2.5% such as from about 0.01% to about 1.0% or from about 0.01% to about 0.1% in weight relative to the total weight of the cosmetic composition. The cosmetic composition can be any composition which can be applied on a skin appendage and/or the scalp. Cosmetic compositions of interest are described herein, further below.

In another aspect, the Invention relates to a cosmetic method for treating skin appendages and/or scalp comprising applying the oak bark extract of the invention or a cosmetic composition comprising the same on the skin appendages and/or the scalp.

The targeted cosmetic effect(s) with the cosmetic method of the invention is/are any of the cosmetic effects described herein such as a seboregulator action on the scalp, a repairing or a regenerating action of skin appendages, an improvement of the appearance or the mechanical properties of the skin appendages such as an increase in shine, strength, mechanical resistance and/or softness of the skin appendages.

In the cosmetic methods and uses according to the Invention, the dose to be administered and the frequency of administration of the extract can vary according to the desired cosmetic effect, the characteristics of the individual, in particular his gender, age and type of skin appendage, especially the hair type.

Typically, the extract can be applied at least once every two weeks at least once a week, even 2 to 3 times a week and even daily on the appendages or the scalp. The extract or the cosmetic composition according to the invention can be applied without rinsing or for at least one minute before rinsing.

By way of example, the extract of the Invention or a cosmetic composition containing it can be applied at least once a week on the hair and/or the scalp.

The cosmetic methods described herein can be performed during several weeks, even several months.

In the cosmetic methods and uses described herein, the oak bark extract may be applied to any subject with hair, namely human beings and furry animals in particular pets such as dogs and cats.

Preferably, the subject is human. The human subject may be of any gender, any ethnic origin or any age. In some embodiments, the subject is at least 6 years old, such as at least 15 years old. In some aspect, the subject is at least 40 years old. In another aspect, the subject is a pre-menopausal or menopausal woman. For instance, the subject is at least 45 years old. In the cosmetic uses and methods described herein, the oak bark extract is preferably from Quercus alba and more preferably a hydroalcoholic extract from Quercus alba bark. In particular embodiments, said extract is obtained by any method described herein, in particular by any method of liquid-solid extraction described herein, e.g. hydroalcoholic extraction.

In the cosmetic uses and methods described herein, the skin appendages are preferably keratin fibers such as eyelashes, eyebrows, beard, mustache, hair and combinations thereof. Preferred keratin fibers are hair, in particular hair which are weakened, stressed and/or damaged because of age or because of an external aggressions or treatments as described herein.

### Cosmetic Products

As mentioned above, the oak bark extract of the Invention is typically applied on the skin appendages or on the scalp in the form of a cosmetic composition.

In an additional aspect, the invention relates to a cosmetic composition comprising an oak bark extract of the Invention. Said cosmetic composition is typically appropriate for use on a skin appendage and/or scalp.

The cosmetic composition typically comprises the oak bark extract of the Invention in an amount of at least or at least about 0.0001%, 0.001%, 0.01%, 0.1%, 1%, 5%, or 10% in weight relative to the total weight of the cosmetic composition.

In some embodiments, the cosmetic composition comprises from 0.0001% to 10%, preferably from 0.001% to 5.0% such as from 0.002% to 2.0% or 0.01% to 1.0% by weight of the oak bark extract preferably as described herein.

The cosmetic composition may also comprise one or more cosmetically acceptable excipients, typically from 50% to 99.9999% by weight, preferably from 60% to 99.999%, for example from 70% to 99.998% by weight, the percentages referring to the total weight of the cosmetic product.

The cosmetically acceptable excipient(s) present in the cosmetic composition can be selected from carriers such as an aqueous solution, demineralized water, mineral, or floral water, solvents, in particular lower alcohols, in particular ethanol, isopropanol, dipropylene glycol, butylene glycol, propanediol, glycerol, pentanediol, hexanediol and propylene glycol, hydroalcoholic mixtures, dispersing agents, gelling or thickening agents, emollients, vectorization agents such as phospholipids or lecithin, preservatives, odor absorbers, pH regulating agents or neutralizing agents, surfactants, in particular anionic, cationic, amphoteric or nonionic surfactants, emulsifying agents, sequestering agents, foam stabilizers, foaming agents, humectants such as glycerin or sorbitol, wetting agents, perfumes, pigments and other coloring agents, opacifying agents, oily agents such as plant oils, silicone oils, fatty acids, fatty alcohols, fatty alcohol esters, paraffins and waxes such as carnauba wax, plasticizing agents, and/or other substances commonly used in formulation in the cosmetic field.

The cosmetic composition according to the invention may also comprise one or more cosmetic active agents, typically in an amount from 0.0001% to 30.0% by weight. The additional cosmetic active agents are selected depending on the intended use. These active agents encompass, without being limited to, UV filters, soothing agents, anti-seborrheic agents, anti-hair loss agents such as hair root stabilizing agents, anti-dandruff agents, moisturizing agents, conditioning agents, antiaging agents, smoothing agents, nourishing agents, antioxidants, anti-pollution agents, anti-itching agent, anti-hair-greying agent and combinations thereof.

For instance, the cosmetic composition can comprise one or several additional active ingredients such as L-carnitine and derivatives thereof, finasteride, Acetyl tetrapeptide-3, Palmitoyl Tetrapeptide-20 Amide plant extracts such as Trifolium pratense flower extract Anetholea Anisata Leaf Extract, Terminalia Ferdinandiana Fruit Extract, Schinus Terebinthifolia Seed Extract, Tasmannia lanceolata fruit/leaf extract Picea Mariana Bark Extract, Epilobium Angustifolium extract, Ilex Paraguariensis Leaf Extract, Alteromonas Ferment Extract, Sunflower seed oil, phospholipids such as sunflower lecithin or Melaleuca Alternifolia oil, vitamins, vitamin precursors or provitamins such as thiamine, pyridoxine, biotin, or panthenol and combinations thereof.

In some embodiments, the cosmetic composition of the invention comprises:
from 0.0001% to 10%, preferably from 0.001% to 5.0% such as from 0.01% to 0.1% by weight of the oak bark extract preferably as described herein,
from 50% to 99.9998%, preferably from 75% to 99.998% such as from 85% to 99.996% of one or several cosmetically acceptable excipients, and
from 0.0001% to 30%, preferably from 0.001% to 20%, more preferably from 0.002 to 10% of one or more additional cosmetic active agents.

The cosmetic composition may be in any form suitable for topical application to the skin appendages or the scalp, in particular to the hair or the scalp.

The cosmetic composition may be in the form of aqueous solutions, dispersions, oil-in-water (O/W) or water-in-oil (W/O) or multiple (e.g. triple: W/O/W or O/W/O), nano-emulsions, micro-emulsions, aqueous gels, dispersions, powder, lacquer, aerosol, spray, oil, serum, foam, this list being not exhaustive.

Typically, the cosmetic composition is in the form of a cosmetic product, preferably a hair care product. The cosmetic product can be a product to be rinsed off or to be leaved-on.

In certain embodiments, the cosmetic composition of the invention is a hair care product. Hair care products of interest encompass, without being limited to, shampoos, including dry or solid shampoos, conditioner, hair masks (e.g. hair nourishing masks), balms, creams, oils, serums, lacquers, fixing gels, hair sprays, perming products, dyeing products, sun or after-sun sprays, oils, foams, this list not being exhaustive.

In other embodiments, the cosmetic product may be selected from makeup products such as mascara, nail polish or vanish, or from shaving or beard care products such as aftershave, foam such as shaving foam, beard wax, oil, balm, shampoo, conditioner and the like.

The following examples are included for illustrative purposes only and are not intended to limit the scope of the invention.

### EXAMPLES

### Example 1: Manufacture of Oak Bark Extract and Characterization

100 g of dried and milled bark from white oak was extracted by a mixture of ethanol and water (1:1 v/v) with a ratio of 1:10 during 1 hour under stirring at 50 °C. Then, after solid/liquid separation by decantation, the liquid phase was filtered until 1 μm.

The liquid phase was then concentrated in order to eliminate the ethanol and spray-dried so as to obtain the exemplary white oak bark extract as a brown powder.

The resulting white oak bark extract was then formulated with a mixture of glycerin/water 4/1 so as to give 360 g of a liquid cosmetic ingredient containing about 2.5% of white oak bark extract by weight.

Before being formulated, the white oak bark extract was analyzed by standard chemical analyses. It contained:
about 18% of total polyphenols expressed in gallic acid determined by the FOLIN-CIOCALTEU method,
about 4% of flavonoids expressed in rutin determined by the AlCl3 method,
about 4.5% of proanthocyanidins expressed in procyanidin B2 determined by the Porter method, and
about 30% of total carbohydrates expressed in glucose, determined by the Phenol method.

### Example 2: Effect on Human Hair Follicular Dermal Papilla Cells (HFDPc) Proliferation

The effect on HFDPc proliferation was evaluated by the PicoGreen method. Quant-iT PicoGreen dsDNA reagent is an ultrasensitive fluorescent nucleic acid stain to quantify double-stranded DNA in solution. The quantity of dsDNA is directly proportional to the number of cells in culture.

5000 cells per well were seeded 96 micro plates in completed medium for 24 hours. The culture medium was then replaced with serum-free Follicle Dermal Papilla Cell Growth Medium for an additional 24 hours. The white oak bark prepared in Example 1 was then added at a concentration of 0.05% by weight in the serum-free medium and the cells were incubated for 24 h. At the end of the incubation period, buffer lysis was added into each well and incubated for 1 hour. dsDNA in solution were then measured using Quant-iT PicoGreen dsDNA kit.

Following supplier indications, the fluorescence of each well was then measured by a plate reader (Excitation 480 nm, Emission 520 nm). The results are indicated as the percentage of proliferation compared to untreated cells (namely cells which were incubated with serum-free Follicle Dermal Papilla Cell Growth Medium without the white oak bark extract) and positive control (cells incubated with the complete medium, namely in the presence of serum).

### Result:

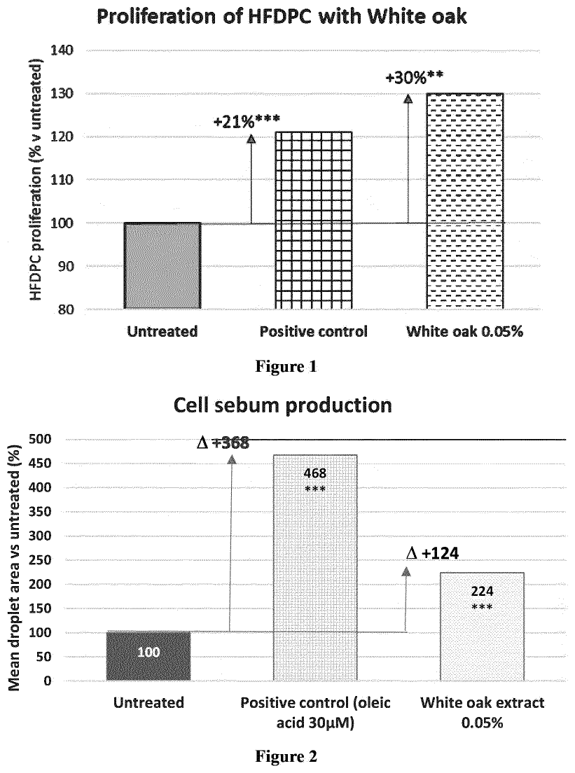

The results are shown in FIG. 1. As expected, medium with serum increased by 21% the proliferation. Surprisingly, after 24 hours of treatment, the white oak bark extract improved significantly the proliferation by 30% at the concentration of 0.05% by weight, compared to the untreated control. Thus, the white oak bark extract of the Invention induced the proliferation of HFDPc.

### Example 3: Experiments on Sebocytes: The White Oak Bark Extract Exerts a Seboregulator Action

The sebocytes were seeded in a 96-well plate at the concentration of 15.000 cells/well in sebocytes culture medium, then allowed to adhere for 24 hours in an incubator at 37 °C, 5% CO₂. After 24 h, the supernatant was removed. These cells were incubated in the following conditions for 24 hours:
Test group: incubation in the presence of the white oak bark extract prepared according to Example 1 (0.05% by weight),
Positive control: incubation with oleic acid (30 μM inducer—positive control)
Untreated group: incubation with the medium only.

At the end of the incubation period, the cells were fixed and stained with a specific lipid dye, BODIPY FL.

Lipid droplets was specifically labeled with Bodipy diluted at 5 μM in PBS. The nuclei were counter-stained using Dapi at 3 μg/mL.

Results were reported as mean fluorescence by cells compared between the untreated and the treated assay.

### Results:

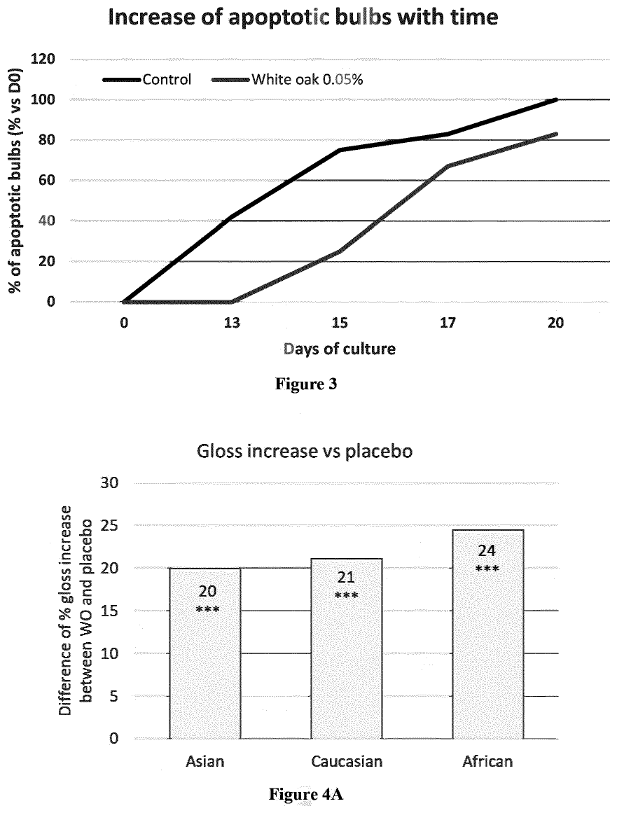

The results are shown in FIG. 2. As expected, oleic acid significantly increased sebum production by 368% compared to untreated cells.

The white oak bark extract at 0.05% by weight also significantly increased sebum production by 124% as compared to untreated cells. Thus, white oak bark extract is able to regulate sebum production in sebocytes.

The White Oak Bark extract was also shown to exert a protective effect against lipid peroxidation and thus to contribute to serum quality. Indeed, the White Oak Bark extract of the Invention was able to inhibit linoleic acid peroxidation in presence of hemoglobin-released oxygen with a IC50 of 0.82% by weight.

### Example 4: In Vitro Anti-Apoptotic Effect on Human Hair Follicular Dermal Papilla Cells (HFDPc)

The aim of this study was to characterize modulation of the pro apoptotic signals in HFDPc after induction with Staurosporine. Signals corresponds to the activation of caspase 3 & 7. The HFDPc were seeded in a 96-well black plate with transparent bottom for imaging at the concentration of 10.000 cells/well, then allowed to adhere for 32 hours in an incubator at 37 °C, 5% CO₂. After 32 h cells products were added in cells supernatant in the presence of Caspase 3&7 probes, with or without Staurosporine inducer.

After cell induction, the 96-well plate was placed on the Cytation 1 reader. The plate reader was maintained at 37 °C and images were acquired with the X4 lens every 30 minutes over 12 hours.

Compared to the apoptotic induction control, the White Oak Bark extract decreased the Caspase 3 & 7 intensity by 22% (p<0.001) and 45% (p<0.001) at 0.05% and 0.1% respectively.

### Example 5: Ex Vivo Model of Human Hair Follicle Culture: The White Oak Bark Extract Increases Hair Longevity

An ex vivo model of hair follicles isolated from human scalp was used to evaluate the effects of the white oak bark extract prepared according to Example 1 on Hair follicle survival. Basically, the hair follicles were treated with the white oak bark extract over a period of 20 days. Hair survival was assessed by counting the number of apoptotic bulbs after 13, 15, 17 and 20 days of incubation. The percentage of apoptosis per condition was calculated by counting the number of apoptotic hair bulbs versus the total number of bulbs.

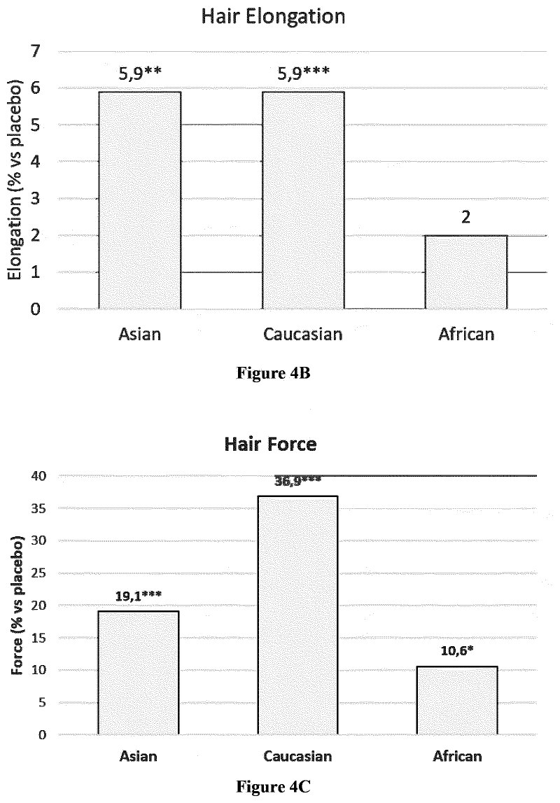

In the control condition, hair follicles started to undergo apoptosis at D13 (42% of apoptotic hair bulbs) and as expected this phenomenon increased throughout the incubation times until reaching 100% of apoptosis at D20. These results were expected and validated the assay. The results are shown in FIG. 3. Under the experimental conditions of the assay, the white oak bark extract, tested at 0.05% by weight, slowed down the entry of hair bulbs into apoptosis: no apoptotic bulb was observed at D13 and a progressive increase of the number of apoptotic bulbs was observed throughout the incubation times: 25% of apoptotic bulbs at D15, 67% at D17 and 83% at D20. Thus, the white oak bark extract of the Invention is able to delay and decrease hair follicle apoptosis and thus increase hair longevity.

### Example 6: Ex Vivo Assay on Damaged Hair Strands: The White Oak Bark Extract Acts as a Repairing Agent Regardless of Hair Type

### Hair Preparation:

The hair locks were artificially damaged by bleaching with H₂O₂. Twenty tresses were prepared from one bleached lock of each hair type: Asian hair type, African hair type and Caucasian hair type.

The tresses weighed 2.5 g each and 25 cm long. All tresses underwent a standard pre-cleaning process with 10% Sodium lauryl ether sulfate (SLES) solution for 1 minute then rinsed with running water. Tresses were dried in a standardized environment at 55±5% relative humidity and 22±2 °C, during 24 hours before tests.

### Treatment:

a) Wet the tress for 20 s and remove excess water.
b) Apply 0.25 mL of the product to test (conditioner with or without white oak bark extract prepared according to Example 1) and rub the tress for 60 seconds. Leave it on hair for 5 minutes. Rinse the tress for 30 seconds and remove excess water.

TABLE 1
Rinsed-off formula used for the test (conditioner) - Test group

| Mixed INCI | % by weight |
|---|---|
| AQUA (WATER) | 74.9 |
| TETRASODIUM EDTA | 0.1 |
| CETEARYL ALCOHOL (AND) CETEARETH-20 | 5.0 |
| CETEARYL ALCOHOL | 4.0 |
| CAPRYLIC/CAPRIC TRIGLYCERIDE | 14.0 |
| PHENOXYETHANOL (AND) METHYLPARABEN (AND) | 0.8 |
| ETHYLPARABEN (AND) BUTYLPARABEN (AND) | |
| PROPYLPARABEN | |
| TOCOPHEROL (AND) HELIANTHUS ANNUUS (SUNFLOWER) | 0.2 |
| SEED OIL | |
| WHITE OAK BARK EXTRACT in glycerin/water | 1.0 |
| (cosmetic ingredient of Example 1) | |

The placebo group was treated with a similar conditioner in which the white oak bark extract was replaced by an equivalent amount of water and glycerin (as the extract is added in the form of the cosmetic ingredient prepared in Example 1).

The effects of the White Oak Bark extract on (i) hair gloss, (ii) hair strength and (iii) hair repairing were assessed as follows:

### Hair Gloss Effect:

The tresses were photographed before the application of the conditioner (Initial) and 24 hours after the application of the conditioner and drying in a controlled environment at 55±5% relative humidity and 22±2 °C (Final).

Digital photographs were obtained for each hair sample. During the image taking process, a photograph tabletop is used, where lighting and distance are controlled.

The digital photographs obtained were analyzed using Image J 1.47v image analysis software. The same reference area was used for the analysis of gloss in the photographs obtained before and after treatment. From the area obtained for the photographs before and after treatment, the digital images were converted to gray scale and binarized.

The Gloss parameter is determined as the percentage in area of black pixels referring to the area obtained after the binarization. Considering that the total number of pixels of the image is constant, the area of black pixels can be directly correlated with the intensity of the Gloss. The larger the observed area, the higher the brightness intensity of the tresses, that is, the higher the gloss intensity.

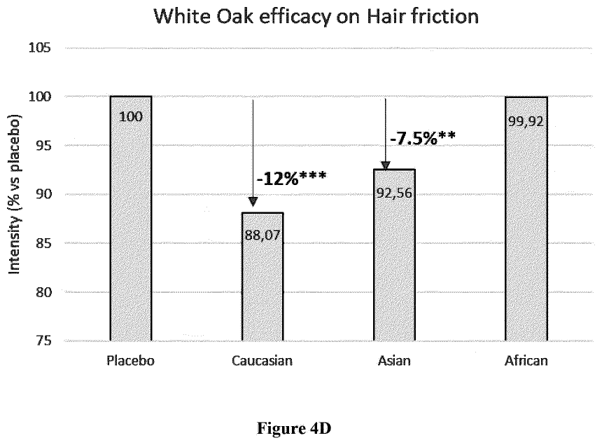

Results obtained with tresses treated with the conditioner containing the extract were compared to the placebo group (tresses treated with the conditioner devoid of white oak bark extract), for each hair type. The results are provided in FIG. 4A.

Whatever the ethnicity, the white oak bark extract significantly increased hair shine from 20% to 24% compared to placebo after one application of a rinse-off product containing 1% of white oak bark extract.

### Hair Strength Effect:

Fifty hair strands were assessed. They were collected from the 3 tresses of each treatment. Each strand was held by a lower claw and an upper claw connected to the load cell of a dynamometer on the upper part. The following parameter was assessed: Elongation at break and Force at 20% of elongation.

The load cell was pre-charged, and the increase in the load was measured with traction speed rates of 100 mm/min. Means and standard deviations were calculated using the GraphPad™ Prism® 6.0 software program.

Results obtained with tresses treated with the conditioner containing the extract were compared to the placebo group (tresses treated with the conditioner devoid of White Oak Bark extract), for each hair type. The results are provided in FIGS. 4B and 4C.

Elongation at breakage is significantly superior to placebo for Asian and Caucasian-type hair (FIG. 4B), and hair strength at 20% elongation is significantly superior to placebo regardless of hair type. Thus, the white oak bark extract of the Invention both increases hair elongation and hair strength (FIG. 4C).

### Repairing of Hair Structure

The hair repairing was assessed using a comb with electronic sensors (software WIDI 4.50 Pro), that are able to traduce de roughness into a melody, from the friction of the fibers against the sensor. Experiments were performed in triplicate. The sound was obtained in the human audible range, between 20 Hz and 20 KHz.

The audio file obtained from the friction of tresses against the equipment was converted on a text file using the software Audacity. Thus, it was possible to obtain data of Intensity (dB) as a function of Frequency (Hz).

Lower Intensity values indicate that the tress showed a lower friction coefficient, which indicates a higher efficiency of the treatment (repairing activity).

Results obtained with tresses treated with the conditioner containing the extract were compared to the placebo group (tresses treated with the conditioner devoid of white oak bark extract), for each hair type.

The tresses treated with white oak bark extract showed significantly lower values of Sound Intensity versus tresses from the placebo group in particular for Asian and Caucasian-type hair. Thus, the treatment with the white oak bark extract of the Invention significantly decreased the friction of the hair, and thus was shown to provide a repairing effect (FIG. 4D).

### Example 7: Ex Vivo Assay on Damaged Hair Strands: Synergic Combination with Sunflower Lecithin on African Hair

### Hair Preparation:

The African-type hair locks were artificially damaged by bleaching with H₂O₂. Twenty tresses were prepared, weighing 2.5 g each and 25 cm long. All tresses underwent a standard pre-cleaning process with 10% Sodium lauryl ether sulfate (SLES) solution for 1 minute then rinsed with running water. Tresses were dried in a standardized environment at 55±5% relative humidity and 22±2 °C, during 24 hours before tests.

### Treatment:

a) Wet the tress for 20 s and remove excess water.
b) Apply 0.25 mL of the product to test (conditioner with or without white oak bark extract prepared in Example 1) and rub the tress for 60 seconds. Leave it on hair for 5 minutes. Rinse the tress for 30 seconds and remove excess water.

TABLE 2
Rinsed-off compositions - test groups and placebo

| | % by weight | | | |
|---|---|---|---|---|
| Mixed INCI | WO | SL | WO + SL | Placebo |
| AQUA (WATER) | 75.07 | 74.94 | 74.91 | 75.10 |
| TETRASODIUM EDTA | 0.1 | 0.1 | 0.1 | 0.1 |
| CETEARYL ALCOHOL (AND) CETEARETH-20 | 5.0 | 5.0 | 5.0 | 5.0 |
| CETEARYL ALCOHOL | 4.0 | 4.0 | 4.0 | 4.0 |
| CAPRYLIC/CAPRIC TRIGLYCERIDE | 14.0 | 14.0 | 14.0 | 14.0 |
| PHENOXYETHANOL (AND) METHYLPARABEN | 0.8 | 0.8 | 0.8 | 0.8 |
| (AND) ETHYLPARABEN (AND) BUTYLPARABEN | | | | |
| (AND) PROPYLPARABEN | | | | |
| TOCOPHEROL (AND) HELIANTHUS ANNUUS | 0.2 | 0.2 | 0.2 | 0.2 |
| (SUNFLOWER) SEED OIL | | | | |
| WHITE OAK BARK EXTRACT (pure) | 0.025 | 0 | 0.025 | 0 |
| Glycerin | 0.8 | 0 | 0 | 0.8 |
| sunflower lecithin(and) sunflower seed | 0 | 0.96 | 0.96 | 0 |
| oil and water (SUNFLOHAIR ™) | | | | |

The effects of the white oak bark extract alone (WO), the sunflower lecithin (Sunflohair™) alone (SL) or in combination with white oak bark (WO+SL) on (i) hair strength and (ii) hair repairing were assessed as follows:

### Hair Strength Effect:

Fifty hair strands were assessed. They were collected from the 3 tresses of each treatment. Each strand was held by a lower claw and an upper claw connected to the load cell of a dynamometer on the upper part. The Force at 20% of elongation was assessed.

The load cell was pre-charged, and the increase in the load was measured with traction speed rates of 100 mm/min. Means and standard deviations were calculated using the GraphPad™ Prism® 6.0 software program.

Results obtained with tresses treated with the conditioner containing the extract alone, the sunflower lecithin alone or combination of both were compared to the placebo group.

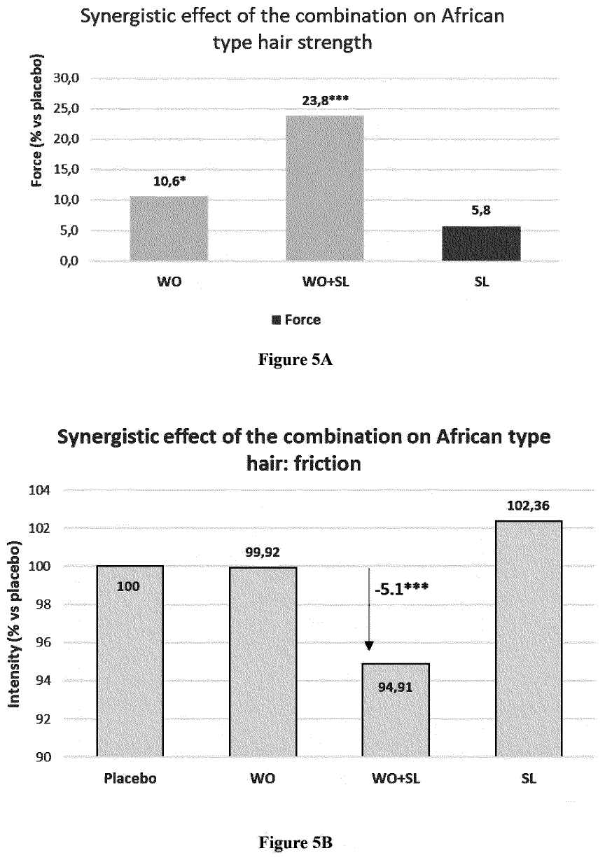

The results are provided in FIG. 5A.

Surprisingly the combination of the White Oak bark extract and the sunflower lecithin (WO+SL) showed a synergistic effect on hair strength, enhancing the force at 20% of elongation by 24% (p<0.001) compared to placebo.

### Repairing of Hair Structure

Hair repairing was assessed as described in Example 6 above.

Results obtained with tresses treated with the conditioner containing the extract alone, sunflower lecithin alone or combination of both were compared to the placebo group.

The results are provided in FIG. 5B.

The tresses treated by the combination of white oak bark and sunflower lecithin (WO+SL) showed significantly lower values of Sound Intensity versus tresses from the placebo group while individual products (WO, SL) did not.

Thus, the treatment with the White Oak Bark extract of the Invention and sunflower lecithin significantly decreased the friction of the hair, and thus was shown to provide a repairing effect even on African-type hair.

### Example 8: Clinical Study

The aim of the study is to evaluate hair regeneration, and hair repairing efficacy of a lotion containing the white oak bark extract prepared according to Example 1 after 120 days of application. The anti-hair loss efficacy, gray/white hair pigmenting efficacy as well as effects on scalp health was also assessed. The following measurements were or will be performed:

Evaluation of Hair Density by TrichoScan® equipment: this is defined as the number of hairs present in a unit of area (generally cm2). Increased hair density at the end of the study demonstrates the efficacy of the regeneration treatment. The number of hairs present by unit of area was calculated from images of the scalp taken with the TrichoScan® microcamera. The computer program associated with TrichoScan® was used for this, permitting manual counting of the number of hairs present in an area equaling 0.592 cm² of scalp. For comparison between the number of hairs present at the end and at the beginning of the study, the increase in the number of hairs in that area was established and hair density could be calculated at the end of the study.

Cosmetic trichogram: it consists of the extraction by traction of a determined number of hairs from the scalp, in order to know in which phase of the hair growth cycle they are in, Anagen growth phase or Telogen fall phase, as well as the initial and final appearance of the hair bulbs. The study is completed by bulb photographs of the three most representative results.

Combing test: the number of hairs falling out during hair combing under standardized conditions is determined. After the regeneration treatment under study, the goal is for the number of hairs falling out during combing to be lower than the initial value. As a precondition, the volunteers come to the visit without having washed their hair at least 48 hours before and without having combed their hair at least 24 hours before, in order to maintain the hairs which are near the end of telogen phase and avoid artificial reduction in the percentage of hairs in telogen phase. The technicians comb each volunteer's hair according with a standard protocol. The hair falling out is collected for later counting.

Wash test: the number of hairs falling out during hair washing under standardized conditions is determined. After the regeneration treatment under study, the goal is for the number of hairs falling out during washing to be lower than the initial value. As a precondition, the volunteers come to the visit without having washed their hair at least 48 hours before and without having combed their hair at least 24 hours before, in order to maintain the hairs which are near the end of telogen phase and avoid artificial reduction in the percentage of hairs in telogen phase. The technicians wash each volunteer's hair according with a standard protocol. The hair falling out is collected for later counting.

Observation with microcamera: The microcamera is a professional diagnostic piece of equipment consisting of a probe for exploring the scalp and the software for capturing and displaying images. The microcamera's function is to allow a direct, magnified view of a hair area to observe its state: number of white hair and/or number of not white hair.

The acceptability of the product is measured additionally, by means of the subjective evaluation of the efficacy, evaluation of the product (organoleptic properties), and mode of application. Additional characteristics of the product will be scored by means of subjective evaluation.

TABLE 3
Evaluated formula containing 0.025%
of the white oak bark extract:

| Mixed INCI | % by weight |
|---|---|
| AQUA (WATER) | 97.30 |
| AMMONIUM ACRYLOYLDIMETHYLTAURATE/VP | 0.50 |
| COPOLYMER | |
| HELIANTHUS ANNUUS (SUNFLOWER) SEED OIL | 0.20 |
| PHENOXYETHANOL (AND) CAPRYLYL GLYCOL | 1.00 |
| Quercus alba bark extract in glycerin/water | 1.00 |
| (cosmetic ingredient of Example 1) | |

TABLE 4
Placebo formula:

| Mixed INCI | % by weight |
|---|---|
| AQUA (WATER) | 97.52 |
| GLYCERIN | 0.78 |
| AMMONIUM ACRYLOYLDIMETHYLTAURATE/VP | 0.50 |
| COPOLYMER | |
| HELIANTHUS ANNUUS (SUNFLOWER) SEED OIL | 0.20 |
| PHENOXYETHANOL (AND) CAPRYLYL GLYCOL | 1.00 |

Product application on scalp for 120 days: Apply daily 5 mL of the product (active or placebo) on the entire scalp, with massage to stimulate microcirculation and comb hair gently, without rinsing.

Instrumental measurements were made at D0, D45 and D120.

Results: The results showed an efficacy significantly better than placebo for hair density (+7.9% versus −1.7% at D45 and +11% versus +4% at D120). At D45, number of hair in anagen phase significantly increased (+6% versus DO for active, −1.3% versus DO for placebo) and number of hair in telogen phase significantly decreased at D120 (−39.3% versus −26.9%), showing a slowdown of the hair cycle. After 120 days of treatment, the white hair proportion amongst the new hair decreased with the White oak bark extract treatment (−32.8% versus −22.4%). After combing and washing, the number of hair loss, at the beginning of the summer period of the clinical study (D120 versus D45, in May-June), was far less important for White oak bark extract treatment (+4%) than for the placebo at the same period (+36%).

The self-assessment showed that after only 45 days of treatment with White oak bark extract, 95.5% of volunteers found that their hair was thicker (vs 81% for placebo), 100% of volunteers found that their hair was more vitalized (vs 95.3%), 95.5% of volunteers found that their scalp was soothed and less itchy (vs 85.7%).

After 120 days of treatment with the White oak bark extract, 95.4% of the volunteers found a reduction in hair loss (versus 85.7% for the placebo), 95.4% found that their hair looked brighter (vs 85.7%), 86.4% that their hair was less grey (vs 80.9%), 100% that their hair appearance has improved (vs 95%), 95.5% that their hair has more volume (vs 90.5%) and 95.4% that their scalp was less itchy (vs 85.7%).

This in vivo study demonstrates that the white oak extract of the invention improves the health of the hair and scalp. The white oak bark extract of the invention increases the hair density by slowing-down hair cycle and decrease the proportion of grey hair. The hair brightness is also increased. The extract of the invention makes the hair look stronger, younger and healthier. The extract of the invention also improves scalp health, in particular by soothing dry scalp.

### Example 9: Examples of Cosmetic Products Containing 0.025% by Weight of White Oak Bark Extract

TABLE 5
Hair Conditioner

| INCI | % by weight |
|---|---|
| AQUA (WATER) | 69.90 |
| GLYCERIN | 4.00 |
| TETRASODIUM EDTA | 0.10 |
| BEHENTRIMONIUM CHLORIDE | 1.00 |
| CETEARYL ALCOHOL (AND) CETEARETH-20 | 5.00 |
| CETEARYL ALCOHOL | 4.00 |
| DIMETHICONE | 2.00 |
| CYCLOPENTASILOXANE (AND) CYCLOHEXASILOXANE | 4.00 |
| CAPRYLIC/CAPRIC TRIGLYCERIDE | 8.00 |
| GLYCERIN (AND) AQUA (WATER) (AND) Quercus alba bark extract | 1.00 |
| (cosmetic ingredient of Example 1) | |
| PHENOXYETHANOL (AND) METHYLPARABEN (AND) | 0.80 |
| ETHYLPARABEN (AND) BUTYLPARABEN (AND) | |
| PROPYLPARABEN | |
| TOCOPHEROL (AND) HELIANTHUS ANNUUS | 0.20 |
| (SUNFLOWER) SEED OIL | |

TABLE 6
Beard serum

| Phase | INCI | % by weight |
|---|---|---|
| | AQUA (WATER) | 92.70 |
| | Tetrasodium EDTA | 0.10 |
| | Xanthan gum (and) Lecithin (and) Sclerotium gum (and) Pullulan | 1.50 |
| | Tocopherol (and) Helianthus annuus (Sunflower) seed oil | 0.20 |
| | Phenoxyethanol (and) caprylyl glycol | 1.00 |
| | GLYCERIN (AND) AQUA (WATER) (AND) QUERCUS ALBA | 1.00 |
| | BARK EXTRACT (cosmetic ingredient of Example 1) | |
| | Glycerin (and) Water (and) Palmitoyl Tetrapeptide-20 | 2.00 |
| | Water (and) Epilobium angustifolium flower/leaf/stem extract | 1.50 |

TABLE 7
Healthy scalp serum

| Phase | INCI | % by weight |
|---|---|---|
| A | AQUA (WATER) | 94.60 |
| A | Tetrasodium EDTA | 0.10 |
| A | Chlorphenesin | 0.30 |
| A | Phenoxyethanol | 0.80 |
| B | Sodium acrylates Copolymer (and) Lecithin | 1.50 |
| C | Tocopherol (and) Helianthus annuus (Sunflower) seed oil | 0.20 |
| D | GLYCERIN (AND) AQUA (WATER) (AND) QUERCUS ALBA | 1.00 |
| | BARK EXTRACT (cosmetic ingredient of Example 1) | |
| D | Water (and) Epilobium angustifolium flower/leaf/stem extract | 1.50 |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in cosmetic field or related fields are intended to be within the scope of the following claims.

1-17. (canceled)

18. A cosmetic ingredient for use in a cosmetic product for skin appendages comprising a water extract or a hydro-alcoholic extract from Quercus alba bark and a vehicle.

19. The cosmetic ingredient according to claim 18, wherein the vehicle is glycerol, C₂-C₅ alkanediols, ketal esters, water, or mixtures thereof.

20. The cosmetic ingredient according to claim 18, wherein the Quercus alba bark extract is obtained by extraction with hydroalcoholic extraction.

21. The cosmetic ingredient according to claim 20, wherein the extraction is performed at a temperature from 30 °C to 70 °C.

22. The cosmetic ingredient according to claim 20, wherein the extraction solvent is water and ethanol in a volume ratio (water to ethanol) from about 0.20 to about 5.0.

23. A cosmetic product for promoting the scalp and/or hair health or for revitalizing the scalp and/or hair, or for improving the visual or mechanical properties of skin appendages comprising a Quercus alba bark extract or the cosmetic ingredient according to claim 18 and one or more cosmetically acceptable ingredients.

24. The cosmetic product according to claim 23, wherein the hair care product is selected from the group consisting of a wet shampoo, a dry shampoo, a conditioner, a balm, a mask, an oil, a serum, a lacquer, a hair fixing gel, a hair spray, a perming product, a hair coloring product and an antiparasitic hair product.

25. A cosmetic method for promoting the health of skin appendages and/or for revitalizing the scalp and/or the hair, and/or for increasing hair density and/or for decreasing or preventing grey hair, and/or for improving the visual or mechanical properties of skin appendages, said method comprising applying an Quercus alba bark extract, a cosmetic ingredient according to claim 18 or a cosmetic product comprising a Quercus alba bark extract or said cosmetic ingredient and one or more cosmetically acceptable ingredients on skin appendages, hair, or scalp.

26. The cosmetic method according to claim 25, wherein the method increases hair density and/or decreases or prevents grey hair.

27. The cosmetic method according to claim 25, wherein the method improves the mechanical or visual properties of skin appendages, hair, or repairing skin appendages.

28. The cosmetic method according to claim 27, wherein said method improves the strength, the resistance, and the shine of skin appendages.

29. The cosmetic method according to claim 27, wherein method repairs the keratin structure of hair.

30. The cosmetic method according to claim 25, wherein the method improves sebum production in dry scalp and/or the scalp suppleness.

31. The cosmetic method according to claim 25, wherein the method alleviates, reduces or treats one or more non-pathological modifications of the skin appendages selected from the group consisting of a loss of thickness, a loss of softness, an increase in dryness, an increase in roughness, a decrease in shiny or silky appearance, a dull or lifeless appearance, a decrease in strength, a decrease in mechanical resistance, brittle skin appendages, a decrease in hair density, a decrease in hair volume, unruly hair or hair more difficult to comb or to shape and combinations thereof.

32. The cosmetic method according to claim 25, wherein the skin appendages are damaged, weakened or stressed hair.

33. The cosmetic method according to claim 25, wherein skin appendages are dyed or bleached hair.
